(19) United States
(12) Patent Application Publication
(10) Pub. No.: US 2026/0055141 A1
(43) Pub. Date: Feb. 26, 2026

(54) COMPOSITIONS AND METHODS FOR CONTROLLING FOOD INTAKE, ENERGY EXPENDITURE, AND BODY WEIGHT FOR THE TREATMENT OF OBESITY AND METABOLIC DISEASES

(71) Applicants: The Trustees of the University of Pennsylvania, Philadelphia, PA (US); Syracuse University, Syracuse, NY (US)

(72) Inventors: Matthew R. Hayes, Lansdale, PA (US); Robert P. Doyle, Manlius, NY (US); Caroline Geisler, West Deptford, NJ (US); Kylie S. Chichura, Norwood, MA (US)

(73) Assignees: The Trustees of the University of Pennsylvania, Philadelphia, PA (US); Syracuse University, Syracuse, NY (US)

(21) Appl. No.: 19/130,833

(22) PCT Filed: Nov. 20, 2023
(86) PCT No.: PCT/US2023/080525

§ 371 Date: May 16, 2025

Related U.S. Application Data
(60) Provisional application No. 63/384,272, filed on Nov. 18, 2022.

(51) Int. Cl. C07K 14/00; A61K 38/00; A61P 3/04

(52) U.S. Cl. CPC C07K 14/00; A61P 3/04; A61K 38/00

(57) ABSTRACT

Compositions and methods for the treatment of obesity and metabolic disorders using an GPR75 inhibitor are provided herein.

### CROSS-REFERENCE TO RELATED APPLICATION

This application claims priority of U.S. Provisional application No. 63/384,272 filed Nov. 18, 2022, the entire contents being incorporated herein by reference as though set forth in full.

### INCORPORATION-BY-REFERENCE OF MATERIAL SUBMITTED IN ELECTRONIC FORM

The Contents of the electronic sequence listing (UPNK-113-PCT.xml; Size: 10,298 bytes; and Date of Creation: Nov. 20, 2023) is herein incorporated by reference in its entirety.

### FIELD OF THE INVENTION

The present invention relates to the fields of weight loss, weight maintenance, obesity, and metabolic disease treatments. More specifically, the invention provides peptide sequences and variants thereof capable of inhibiting G-Protein Receptor 75 (GPR75).

### BACKGROUND OF THE INVENTION

Several publications and patent documents are cited throughout the specification in order to describe the state of the art to which this invention pertains. Each of these citations is incorporated by reference herein as though set forth in full.

Obesity and its cardio-metabolic complications, particularly type 2 diabetes and coronary artery disease, account for significant morbidity and mortality globally. There is a substantial unmet medical need for safe and effective weight loss approaches and strategies for maintaining the weight-reduced state.

The implementation of lifestyle interventions, such as diet and physical activity, is the first option for the management of obesity, but efficacy can be limited, and weight regain is common. Bariatric surgery can be highly effective for weight loss in severely obese or high-risk patients, but its use is limited by its invasive nature, cost, and risk of perioperative adverse events including perioperative death. While a few drugs have demonstrated efficacy in weight-reduction, pharmacotherapy for the treatment of obesity is limited by the modest weight loss induced by most drugs, the development of dependency on said drugs, side effect profiles, contraindications, low compliance, and societal barriers to treatment.

G-Protein Receptor 75 (GPR75) is a member of the G protein-coupled receptor family. GPRs are cell surface receptors that activate guanine-nucleotide binding proteins upon the binding of a ligand. GPR75 is likely coupled to heterotrimeric Gq proteins and stimulates inositol trisphosphate production and calcium mobilization upon activation. Various experiments have shown a strong association of GPR75 with obesity and metabolic diseases; however, no known molecules exist that antagonize GPR75. Clearly, GPR75 inhibitors for the treatment of obesity and metabolic diseases are urgently needed.

### SUMMARY OF THE INVENTION

In accordance with present invention, isolated or purified peptides of SEQ ID NO: 1 or SEQ ID NO: 2, sequences having at least 95% identity to SEQ ID NOS: 1 or 2 and functional fragments thereof are provided for effective weight management and the treatment of obesity.

In certain embodiments, the peptides have an anti-obesity activity. In certain embodiments, the peptides comprise one or more modified amino acids selected from Gln(alkyn), Ala(Alkyn), Gly(Alkyn), Lys(N3) and the modified amino acids listed in Table 2 for bioconjugation including lipidation and/or fluorescent tagging. In certain embodiments, the modified amino acid facilitates bioconjugation of an agent selected from one or more of a N and, or C terminus protection moiety, a lipid, a recombinant FC-peptide, a cell penetrating peptide, and enhances one or more of peptide function, stability, and bioavailability. In a preferred embodiment, the peptides are delivered in a pharmaceutically acceptable carrier. In certain embodiments, the composition further comprises at least one of a HEPES buffer and an acetate salt. In certain embodiments, the composition is formulated to retain a neutral pH. In certain embodiments, the composition is formulated for intracerebroventricular injection or peripheral administration.

In another aspect, isolated nucleic acids encoding amino acid sequences of SEQ ID NOS: 1 and SEQ ID NO: 2 are also disclosed. In certain embodiments, the nucleic acid is encapsulated in a liposome or an extracellular vesicle or affixed to a nanoparticle or lipid particle. In other aspects, the isolated nucleic is present in a vector for robust expression and production in an organism of interest. In certain embodiments, the vector is a plasmid vector, a lentiviral vector, or an AAV vector. In certain embodiments, the vector is encapsulated in a liposome or an extracellular vesicle or affixed to a nanoparticle or lipid nanoparticle. Administration of said peptides, nucleotides, vectors, or compositions can be via any suitable route, e.g., systemically, intramuscular, topical, oral, parenteral, transdermal patch, aerosolized, pulmonary, ophthalmic, buccal, and lingually.

In yet another embodiment, a method of treating obesity in a subject in need thereof comprises administering an effective amount of the peptides described above is disclosed.

Also provided is a method of treating a metabolic disease or disorder in a subject in need thereof, the method comprising administering an effective amount of the peptide of SEQ ID NO: 1 or SEQ ID NO: 2 or functional variants thereof. Metabolic diseases or disorders to be treated include, without limitation, obesity, diabetes mellitus, dyslipidemia, insulin resistance, hepatic steatosis, hypercholesterolemia, and non-alcoholic fatty liver. In certain aspects, the disease is diabetes mellitus selected from type 1 or type 2 diabetes. In other aspects, the method can further comprise administering a second therapeutic agent that treats or inhibits obesity. In certain embodiments, the methods further comprise enforcing lifestyle interventions on diet and/or physical activity on the subject. In preferred embodiments, the weight of the patient decreases following administration of the peptide.

In certain embodiments, the patient has a reduced food intake for at least 1, 3, 6, and/or 24 hours after administration of said peptide when compared to an untreated control. In another aspect, the method can further comprise assessing the patient for a reduction in obesity symptoms. In certain embodiments, the patient is assessed for a reduction in symptoms of obesity, diabetes mellitus, dyslipidemia, insulin resistance, hepatic steatosis, hypercholesterolemia, or non-alcoholic fatty liver. In certain embodiments, the peptides, nucleotides, vectors, or compositions can be administered via any suitable route, e.g., systemically, intramuscular, topical, oral, parenteral, transdermal patch, aerosolized, pulmonary, ophthalmic, buccal, and lingually. In certain embodiments, the administration is via intracerebroventricular injection or peripheral administration.

In certain embodiments, the peptide and second therapeutic agent act synergistically to increase weight loss.

### BRIEF DESCRIPTION OF THE DRAWINGS

FIGS. 1A-1B: Confirmation of synthesis and purity of peptides. (FIG. 1A) SU75-36 (4894.1 g/mol; 17.084 min TR; 100% purity, (FIG. 1B) SU75-37 (4462.9 g/mol; 15.381 min TR; 98.3% purity).

FIG. 2: Blind SU75-36/GPR75 receptor in silico docking using the HPEPDOCK program. SU75-36 (aqua marine; see arrow) surface binding of GPR75 consistent with Surface Plasmon Resonance (SPR) binding (FIG. 3). Docking score 0.884.

FIG. 3: Surface Plasmon Resonance (SPR) assays tracking SU75-36 binding at GPR75. SU75-36 binds to GPR75 with a K_{D} of 7.76 μM.

FIG. 4: Surface Plasmon Resonance (SPR) assays tracking SU75-37 binding at GPR75. SU75-37 binds to GPR75 with a K_{D} of 23.8 μM.

FIG. 5: Surface Plasmon Resonance (SPR) assays comparing SU75-36 binding at hGLP-1R with positive Ex-4 control, negative ODN control, and SU75-37. SU75-37 does not bind at the hGLP-1R.

FIG. 6: Surface Plasmon Resonance (SPR) assays tracking SU75-36 binding at hGLP-1R. SU75-36 binds to hGLP-1R with a K_{D} of 182 μM.

FIGS. 7A-7B: 4th Ventricle GPR75 Ligands Suppress Food Intake and Body Weight in High Fat Diet (HFD) Rats. Effect of 4th ventricle SU75-36 (20, 100, or 200 μg/2 μL in aCSF) and SU75-37 (20 μg/2 μL in aCSF) treatment on 24 h food intake (FIG. 7A) and body weight change (FIG. 7B) in HFD fed rats. All data presented as mean±SEM.

FIGS. 8A-8F: Lateral Ventricle GPR75 Ligands injection suppresses food intake and body weight in Chow and HFD Rats. Effect of lateral ventricle SU75-36 (20 or 100 μg/2 μL in aCSF) or SU75-37 (20 μg/2 μL in aCSF) treatment on 24 h food intake in chow (FIG. 8A) and HFD fed rats (FIG. 8B), kaolin intake in chow (FIG. 8C) and HFD fed rats (FIG. 8D) and body weight change in chow (FIG. 8E) and HFD fed rats (FIG. 8F). All data presented as mean±SEM.

FIG. 9: Secondary structure analysis of SU75-36 and SU75-37 by CD Spectroscopy at 40 μM in 0.5% saline. Percent helicity was determined to be 20.9% and 21.3% for SU75-36 and SU75-37, respectively.

FIGS. 10A-10B: Central 36 suppresses 24 h food intake in rats in 4-(2-hydroxyethyl)-1-piperazineethanesulfonic acid (HEPES) buffer. Effect of lateral ventricle IV administration of 36 (150 μg/3 μL) on 24 h food intake (FIG. 10A) and body weight (FIG. 10B) in male HFD fed rats following treatments. All data presented as mean±SEM.

FIGS. 11A-11B: Central optimized 37 suppresses 24 h food intake in rats in HEPES buffer. Effect of lateral ventricle IV administration of 37 (150 μg/3 μL) on 24 h food intake (FIG. 11A) and body weight (FIG. 11B) in male HFD fed rats following treatments. All data presented as mean±SEM.

FIGS. 12A-12D: Peripheral 36 and 37 suppresses 24 h food intake in mice. Effect of IP 36 (0.5 and 5 mg/kg) on 24 h food intake (FIG. 12A) and body weight (FIG. 12B) and 37 (0.5 and 5 mg/kg) on 24 h food intake (FIG. 12C) and body weight (FIG. 12D) in male HFD fed mice following treatments. All data presented as mean±SEM.

FIGS. 13A-13B: Peripheral optimized 37 suppresses 24 h food intake in mice. Effect of IP 37 (10 mg/kg) on 24 h food intake (A) and body weight (B) in male HFD fed mice following treatments. All data presented as mean±SEM.

### DETAILED DESCRIPTION OF THE INVENTION

As demonstrated herein, we provide novel, non-naturally occurring peptide ligands that antagonize the highly sought-after orphan receptor GPR75. These GPR75 inhibitors represent a major drug discovery in the pharmaceutical industry given the association of GPR 75 with obesity and metabolic diseases.

### Definitions

Unless otherwise defined herein, scientific and technical terms used in connection with the present application shall have the meanings that are commonly understood by those of ordinary skill in the art. In addition to definitions included in this sub-section, further definitions of terms are interspersed throughout the text.

In this invention, “a”, “or” and “an” can mean “at least one” or “one or more,” etc., unless clearly indicated otherwise by context. The term “or” means “and/or” unless stated otherwise. In the case of a multiple-dependent claim, however, use of the term “or” refers back to more than one preceding claim in the alternative only.

Furthermore, a compound “selected from the group consisting of” refers to one or more of the compounds in the list that follows, including mixtures (i.e. combinations) of two or more of the compounds. According to the present invention, an isolated, or biologically pure molecule is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

The terms “agent”, and “test compound” denote a chemical compound, a mixture of chemical compounds, a biological macromolecule, or an extract made from biological materials such as bacteria, plants, fungi, or animal (particularly mammalian) cells or tissues. Biological macromolecules include peptides, peptide/DNA complexes, siRNA, shRNA, antisense oligonucleotides, and any nucleic acid-based molecule which encoded the proteins described herein.

It is also contemplated that the term “compound” or “compounds” refers to the compounds discussed herein and includes precursors and derivatives of the compounds, and pharmaceutically acceptable salts of the compounds, precursors, and derivatives.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the functional and novel characteristics of the sequence.

A “derivative” of a polypeptide, polynucleotide or fragments thereof means a sequence modified by varying the sequence of the construct, e.g., by manipulation of the nucleic acid encoding the protein or by altering the protein itself. “Derivatives” of a gene or nucleotide sequence refers to any isolated nucleic acid molecule that contains significant sequence similarity to the gene or nucleotide sequence or a part thereof. In addition, “derivatives” include such isolated nucleic acids containing modified nucleotides or mimetics of naturally-occurring nucleotides.

The term “functional” as used herein implies that the nucleic or amino acid sequence is functional for the recited assay or purpose.

For purposes of the invention, “nucleic acid”. “nucleotide sequence” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. Alternatively, this term may refer to a DNA that has been sufficiently separated from (e.g., substantially free of) other cellular components with which it would naturally be associated.

“Isolated” is not meant to exclude artificial or synthetic mixtures with other compounds or materials, or the presence of impurities that do not interfere with the fundamental activity, and that may be present, for example, due to incomplete purification. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (cither DNA or RNA) may further represent a molecule produced directly by biological or synthetic means and separated from other components present during its production.

A “specific binding pair” comprises a specific binding member (sbm) and a binding partner (bp) which have a particular specificity for each other and which in normal conditions bind to each other in preference to other molecules. Examples of specific binding pairs are antigens and antibodies, biotin and streptavidin, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long.

According to the present invention, an isolated or biologically pure molecule or cell is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

The term “delivery” as used herein refers to the introduction of foreign molecule (i.e., miRNA encoding the polypeptide of interest) into cells. The term “administration” as used herein means the introduction of a foreign molecule into a cell. The term is intended to be synonymous with the term “delivery”.

### Peptides

The peptides of the invention inhibit or modulate GPR75 activity. The terms “inhibition” or “inhibit” refer to a decrease or cessation of any event (such as protein ligand binding) or to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. It is not necessary that the inhibition or reduction be complete. For example, in certain embodiments, “reduce” or “inhibit” refers to the ability to cause an overall decrease of 20% or greater. In another embodiment, “reduce” or “inhibit” refers to the ability to cause an overall decrease of 50% or greater. In yet another embodiment, “reduce” or “inhibit” refers to the ability to cause an overall decrease of 75%, 85%, 90%, 95%, or greater.

The term “modulate” as used herein refers to the ability of a compound to change an activity in some measurable way as compared to an appropriate control. As a result of the presence of compounds in the assays, activities can increase or decrease as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound. A compound that increases a known activity is an “agonist”. One that decreases, or prevents, a known activity is an “antagonist.”

The term “inhibitor” refers to an agent that slows down or prevents a particular chemical reaction, signaling pathway or other process, or that reduces the activity of a particular reactant, catalyst, or enzyme.

The phrase “G-Protein Receptor 75” or “GPR75” refers to a member of the G protein-coupled receptor family. GPRs are cell surface receptors that activate guanine-nucleotide binding proteins upon the binding of a ligand. GPR75 is a protein coding gene. Among its related pathways are Class A/1 (Rhodopsin-like receptors) and 15q13.3 copy number variation syndrome. Gene Ontology (GO) annotations related to this gene include G protein-coupled receptor activity and C-C chemokine receptor activity.

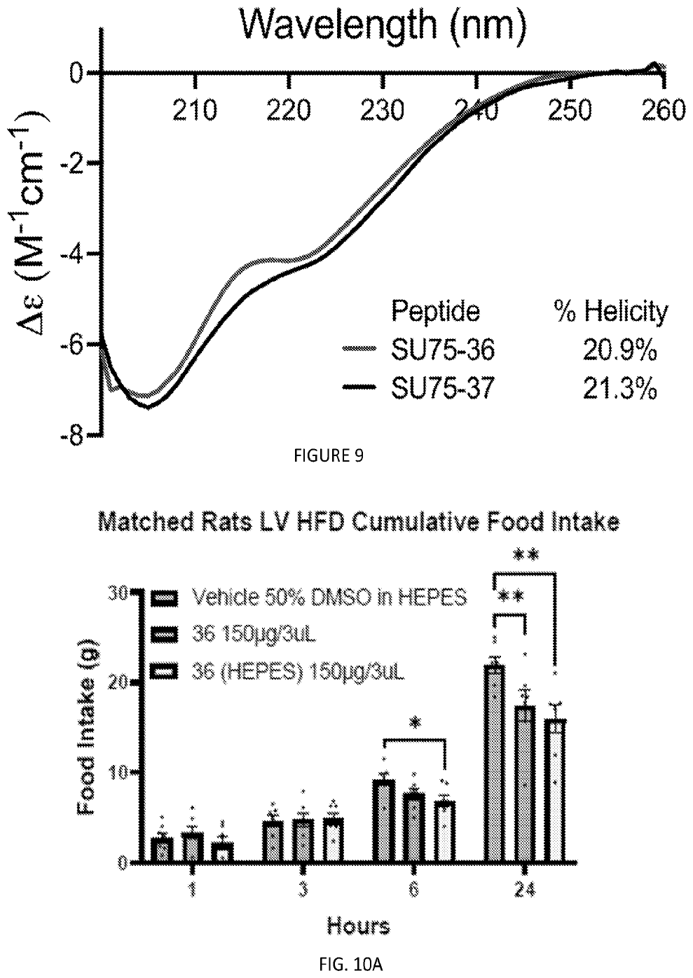

The phrase “G-Protein Receptor 75 inhibitor” or “GPR75 inhibitor” refers to a class of agents that inhibit the action of GPR75. The peptides of interest herein are GPR75 inhibitors which each adopt an α-helical secondary structure (FIG. 9). Exemplary GPR75 binding peptides are provided in Table 1.

TABLE 1
GPR75 Binding Peptides

| | | SEQ ID |
|---|---|---|
| Peptide | Sequence | NO |
| SU75-36 | HsQGTFTSDLSKYLEEEVREFIWLKNGGPSDVNTDRPGLLDLK-NH₂ | 1 |
| SU75-37 | TFTSDLSKYLEEEVREFIWLKNGGPSDVNTDRPGLLDLK-NH₂ | 2 |

In certain embodiments, the residues of the protein or peptide are sequential, without any non-genetically encoded amino acids, or synthetic amino acids interrupting the sequence of amino acid residues. In other embodiments, the sequence may comprise one or more non-genetically encoded or synthetic amino acid moieties. In particular embodiments, the sequence of residues of the peptide may be interrupted by one or more non-genetically encoded or synthetic amino acid moieties, including but not limited to those shown in Table 2.

TABLE 2
Non-genetically Encoded or Synthetic Amino Acids

| Abbreviation | Amino Acid | Abbreviation | Amino Acid |
|---|---|---|---|
| AcLys | N-Acetyl lysine | 4FPhe | 4-Fluorophenylalanine |
| Aad | 2-Aminoadipic acid | Hip | Hippuric acid |
| Baad | 3-Aminoadipic acid | hArg | Homoarginine |
| Bala | β-alanine, 3-Amino-propionic | hCys | Homocysteine |
| | acid | | |
| Abu | 2-Aminobutyric acid | hSer | Homoserine |
| 4Abu | 4-Aminobutyric acid, | AHyl | allo-Hydroxylysine |
| | piperidinic acid | | |
| Aha | 6-Aminohexaboic acid | 3.Hyp | 3-Hydroxyproline |
| Ahe | 2-Aminoheptanoic acid | 4Hyp | 4-Hydroxyproline |
| Aib | 2-Aminoisobutyric acid | Hyl | Hydroxylysine |
| Baib | 3-Aminoisobutyric acid | Ide | Isodesmosine |
| Apa | 5-Aminopentoic acid | AIle | allo-Isoleucine |
| pAmPhe | p-Aminophenylalanine | MSO | Methionine sulfoxide |
| Apm | 2-Aminopimelic acid | MeAla | 2-Methyl-alanine |
| Dap | 3-Aminopropionic acid | MeGly | N-Methylglycine, sarcosine |
| BztAla, B | 3-Benzothiazol-2-yl-alanine | MeIle | N-Methylisoleucine |
| BzPhe | para-Benzoyl-phenylalanine | MeLys | 6-N-Methyllysine |
| tBuA | t-Butylalanine | MeVal | N-Methylvaline |
| tBuG | t-Butylglycine | Nal | 2-Naphthylalanine |
| cGlu | gamma-Carboxyglutamate | Nwa | Norvaline |
| Cit | Citrulline | Nle, J | Norleucine |
| 4ClPhe | 4-Chlorophenylalanine | Oct | Octahydro-1H-indole-2-carboxylic |
| | | | acid |
| Cha | Cyclohexylalanine | Orn | Ornithine |
| Dab | 2,3-Diaminobutyric acid | Pen | Penicillamine |
| Dbu | 2,4-Diaminobutyric acid | Phg | 2-Phenylglycine |
| Des | Desmosine | pSer | Phosphoserine |
| Dpm | 2,2′-Diaminopimelic acid | pThr | Phosphothreonine |
| Dpr, Z | 2,3-Diaminopropionic acid | pTyr | Phosphotyrosine |
| EtAsn | H-Ethylasparagine | PropGly | Propargylglycine |
| EtGly | N-Ethylglycine | Sta | Statine |
| 2FPhe | 2-Fluorophenylalanine | Tic | 1,2,3,4-Tetrahydroisoquinoline-3- |
| | | | carboxylic acid |
| 3FPhe | 3-Fluorophenylalanine | Thi | beta-2-Thienylalanine |

In certain embodiments, the peptides disclosed herein may be modified to optimize peptide function, stability, formulation and or solubility using the approaches described in Table 3.

TABLE 3
Structural modification strategies to optimize peptide function and/or stability and/or
formulation/solubility. AA = amino acid. Fc = ‘Fragment crystallizable’ of IgG.

| General Stability/ | |
|---|---|
| Clearance Approach | Examples |
| N-/C- termini protection | N-acetylation and C-amidation |
| Use of D-isomer and/or | Replacement of AAs at sites of proteolysis |
| non-canonical AA | |
| Conjugation to | Lipidation e.g. (C15-C20 carboxylic acid capped) at C- or |
| macromolecules | N-termini and/or sites that do not remove function as screened |
| | in vitro. |
| Fusion technology | Recombinant Fc-Peptide, CPP, targeting tag (e.g., ligand for |
| | the transferrin receptor) |

These modifications will produce analogs with prolonged half-lives allowing, for example, once weekly dosing.

In certain embodiments, the present invention includes peptides that have at least 80% identity to anyone of the peptides described herein. The phrases “% sequence identity,” “percent identity.” or “% identity” refer to the percentage of residue matches between at least two amino acid sequences aligned using a standardized algorithm. Methods of amino acid sequence alignment are well-known. Some alignment methods take into account conservative amino acid substitutions. Such conservative substitutions generally preserve the charge and hydrophobicity at the site of substitution, thus preserving the structure (and therefore function) of the polypeptide. Percent identity for amino acid sequences may be determined as understood in the art. In certain embodiments, the peptides of the invention have a sequence identity of at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity.

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, may be used to describe a length over which percentage identity may be measured.

Preferably, the peptides of the above-described sequences and functional equivalents thereof which act to modulate obesity upon administration. As used herein, the term “functional equivalent” is intended to include amino acid sequence variants having amino acid substitutions in some or all of the proteins, or amino acid additions or deletions in some of the proteins. The amino acid substitutions are preferably conservative substitutions. Examples of the conservative substitutions of naturally occurring amino acids are as follow: aliphatic amino acids (Gly. Ala, and Pro), hydrophobic amino acids (Ile, Leu, and Val), aromatic amino acids (Phe. Tyr, and Trp), acidic amino acids (Asp, and Glu), basic amino acids (His, Lys, Arg. Gln, and Asn), and sulfur-containing amino acids (Cys, and Met). The deletions of amino acids are preferably located in a region which is not directly involved in the activity of the peptide.

In the present context, the term “variant” refers to a nucleic acid sequence or polypeptide comprising a sequence, which differs (by deletion, insertion, and/or substitution of a nucleic acid or amino acid, an L or D stereoisomer an amino acid, or a non-naturally occurring amino acid) in one or more nucleic acid or amino acid positions differ from that of a wild type nucleic acid or polypeptide sequence.

In the present context, the term “linker” refers to a connection between two protein coding sequences or their protein products. Linkers comprise a stretch of contiguous nucleic acids or amino acids, which holds at least one cleavage site that enables separation of the genes or their products through cleavage of the linker. Preferably, the linker comprises a cleavage site at its 5′ end and a cleavage site at its 3′ end, or a cleavage site at its N-terminal end and a cleavage site at its C-terminal end.

The peptide may be fused to biotin, Poly-lysine, lysozyme. Green fluorescent protein (and derivatives), SUMO or other desired proteinaceous tags. Production of the desired peptide sequence can be carried out in E. coli, SF9, Pichia, etc., using existing technologies, e.g. with protein fusion tags that can either be removed or left as desired. In certain embodiments, the peptide of interest may be fused via a linker.

The peptide can be fused to one or more cell penetrating peptides (CPP) which are useful for facilitating delivery of the peptide into target cells. CPPs are known to the skilled person and include without limitation, penetratin (RQIKIWFQNRRMKWKK) (SEQ ID NO: 3), VP22 peptide (DAATATRGRSAASRPTER PRAPARSASRPRRVD) (SEQ ID NO: 4), MAP (KLALKLALKALKAALKLA-amide) (SEQ ID NO: 5), Transportin (GWTLNSAGYLLGKINLKALAALAKKIL-amide) (SEQ ID NO: 6) R7 (RRRRRRR) (SEQ ID NO: 7), MPG (GALFLGWLGAAGSTMGAPKKRKV) (SEQ ID NO: 8), and Pep-1 (KETWWETWWTEWSQPKKKRKV) (SEQ ID NO: 9) and t at (YGRKKRRQRRR; SEQ ID NO: 10).

The peptide can be expressed as a fusion to larger proteins, facilitating expression at large scales, ease of purification, and ensuring quality of product. Expression systems can also be leveraged to generate large sequence libraries, allowing for directed evolution for targeted properties. Peptides can be produced sustainably using environmentally friendly, existing fermentation technologies.

As noted above, the invention also includes polynucleotides encoding the peptides or fusion proteins comprising the peptide described herein. Those of skill in the art understand the degeneracy of the genetic code and that a variety of polynucleotides can encode the same polypeptide. In some embodiments, the polynucleotides (i.e., polynucleotides encoding the fusion polypeptides) may be codon-optimized for expression in a particular cell including, without limitation, a plant cell, bacterial cell, or algal cell. Any polynucleotide sequences may be used which encode a desired form of the polypeptides described herein. The polynucleotide sequences which encode the polypeptides of the invention represent non-naturally occurring sequences. Computer programs for generating degenerate coding sequences are available and can be used for this purpose.

In the present context, the term “codon optimization” refers to changing the codons of a nucleotide sequence without altering the amino acid sequence that it encodes in order to favor expression in a specific species. Codon optimization may be used to increase the abundance of the peptide or protein that the nucleotide sequence encodes since “rare” codons are removed and replaced with abundant codons.

Regarding the fusion polypeptides disclosed herein, the structural similarity is typically at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity.

### In Vitro Synthesis of Peptides

Peptides can be synthesized chemically either in solution or on a solid phase. The process involves directed and selective formation of an amide bond between an N-protected amino acid and an amino acid bearing a free amino group and protected carboxylic acid. In solid phase synthesis, the carboxyl protecting group is linked to a polymer support. Following bond formation, the amino-protecting group of the dipeptide is removed, and the next N-protected amino-acid is coupled.

Solid-phase peptide synthesis (SSPS) is the most frequently used method of peptide synthesis due to its efficiency, simplicity, speed, and ease of parallelization. SPPS involves sequential addition of amino and side-chain protected amino acid residues to an amino acid or peptide attached to an insoluble polymeric support. Either an acid-labile Boc group (Boc SPPS) or base-labile Fmoc-group (Fmoc SPPS) is used for N-α-protection. After removal of this protecting group, the next protected amino acid is added using either a coupling reagent or pre-activated protected amino acid derivative. The C-terminal amino acid is anchored to the resin via a linker, the nature of which determines the conditions required to release the peptide from the support after chain extension. Side-chain protecting groups are often chosen so as to be cleaved simultaneously with detachment of the peptide from the resin.

Peptides of 50 amino acids can be routinely prepared although the synthesis of proteins of over 100 amino acids are commonly reported. Longer proteins can be made by native chemical ligation of fully deprotected peptides in solution. With this method, it is possible to synthesize natural peptides that are difficult to express in bacteria, to incorporate unnatural or D-amino acids, and to generate cyclic, branched, labelled, and post-translationally modified peptides.

Liquid-phase peptide synthesis, usually utilizing Boc or Z-amino protection, has been superseded by SPPS except for existing processes of large-scale synthesis of peptides for industrial purposes. Desired sequences can be developed by any one of the several commercial entities who provide this service for a fee, including Sigma Aldrich, and Avivasysbio for example.

### Vectors and Production

Transgenic cells expression of said polynucleotides and polypeptides also forms an aspect of the invention. A transgenic cell may be obtained by introducing a recombinant nucleic acid molecule that encodes a protein of this disclosure. As used herein, the term “recombinant nucleic acid” refers to a polynucleotide that is manipulated by human intervention. A recombinant nucleic acid molecule can contain two or more nucleotide sequences that are linked in a manner such that the product is not found in a cell in nature. In particular, the two or more nucleotide sequences can be operatively linked and, for example, can encode a fusion polypeptide. A recombinant nucleic acid molecule also can be based on, but manipulated so as to be different, from a naturally occurring polynucleotide, for example, a polynucleotide having one or more nucleotide changes such that a first codon, which normally is found in the polynucleotide, is biased for chloroplast codon usage, or such that a sequence of interest is introduced into the polynucleotide, for example, a restriction endonuclease recognition site or a splice site, a promoter, a DNA origin of replication, or the like.

Any appropriate technique for introducing recombinant nucleic acid molecules into cells may be used. Techniques for nuclear and chloroplast transformation are known and include, without limitation, electroporation, biolistic transformation (also referred to as micro-projectile/particle bombardment), agitation in the presence of glass beads, and Agrobacterium-based transformation.

As used herein, the term “construct” refers to recombinant polynucleotides including, without limitation, DNA and RNA, which may be single-stranded or double-stranded and may represent the sense or the antisense strand. Recombinant polynucleotides are polynucleotides formed by laboratory methods that include polynucleotide sequences derived from at least two different natural sources or they may be synthetic. Constructs thus may include new modifications to endogenous genes introduced by, for example, genome editing technologies. Constructs may also include recombinant polynucleotides created using, for example, recombinant DNA methodologies.

A “vector” is capable of transferring gene sequences to target cells. Typically, “vector construct.” “expression vector,” and “gene transfer vector,” mean any nucleic acid construct capable of directing the expression of a gene of interest and which can transfer gene sequences to target cells. Thus, the term includes cloning and expression vehicles, as well as integrating vectors.

The constructs and vectors provided herein may be prepared by methods available to those of skill in the art. Notably each of the constructs or expression cassettes claimed are recombinant molecules and as such do not occur in nature. Generally, the nomenclature used herein and the laboratory procedures utilized in the present invention include molecular, biochemical, and recombinant DNA techniques that are well known and commonly employed in the art. Standard techniques available to those skilled in the art may be used for cloning, DNA and RNA isolation, amplification and purification. Such techniques are thoroughly explained in the literature.

The constructs and expression cassettes provided herein may include a promoter operably linked to any one of the polynucleotides described herein, but need not, have a promoter and may be used for homologous recombination into the cell. The constructs may include a promoter and the promoter may be a heterologous promoter or an endogenous promoter associated with the polypeptide.

As used herein, the terms “heterologous promoter.” “promoter.” “promoter region,” or “promoter sequence” refer generally to transcriptional regulatory regions of a gene, which may be found at the 5′ or 3′ side of the polynucleotides described herein, or within the coding region of the polynucleotides, or within introns in the polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

In some embodiments, the disclosed polynucleotides are operably connected to the promoter. As used herein, a polynucleotide is “operably connected” or “operably linked” when it is placed into a functional relationship with a second polynucleotide sequence. For instance, a promoter is operably linked to a polynucleotide if the promoter is connected to the polynucleotide such that it may affect transcription of the polynucleotides. In various embodiments, the polynucleotides may be operably linked to at least 1, at least 2, at least 3, at least 4, at least 5, or at least 10 promoters.

Heterologous promoters useful in the practice of the present invention include, but are not limited to, constitutive, inducible, temporally-regulated, developmentally regulated, chemically regulated, tissue-preferred and tissue-specific promoters. The heterologous promoter may be a plant, animal, bacterial, fungal, or synthetic promoter.

### Methods of Treatment and Administration

The term “reducing” or “inhibiting” as used herein refers to administering a compound prior to, or during the onset of clinical symptoms of a disease or conditions so as to reduce a physical manifestation of aberrations associated with the disease or condition.

The term “in need of treatment” as used herein refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that includes the knowledge that the subject is ill, or will be ill, as the result of a condition that is treatable by the disclosed compounds.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

As used herein. “subject” includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird, a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

In some embodiments, treatment produces a measurable therapeutic effect comprising one or more of a decrease/reduction in obesity, a decrease/reduction in the severity of obesity (such as, for example, a reduction or inhibition of development or obesity), a decrease/reduction in symptoms and obesity-related effects, delaying the onset of symptoms and obesity-related effects, reducing the severity of symptoms of obesity-related effects, reducing the severity of an acute episode, reducing the number of symptoms and obesity-related effects, reducing the latency of symptoms and obesity-related effects, an amelioration of symptoms and obesity-related effects, reducing secondary symptoms, reducing secondary infections, preventing relapse to obesity, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of obesity development/progression (such as, for example, a complete or partial avoidance/inhibition or a delay), or an increased survival time of the affected host animal, following administration of a therapeutic protocol. Treatment of obesity encompasses the treatment of subjects already diagnosed as having any form of obesity at any clinical stage or manifestation, the delay of the onset or evolution or aggravation or deterioration of the symptoms or signs of obesity, and/or preventing and/or reducing the severity of obesity.

In another aspect, provided herein are methods for treating a patient comprising administration of the peptides of interest. In any of the embodiments described herein, the subject can be obese, or have excessive weight, elevated BMI, elevated body fat mass, percentage, or volume, and/or excessive food intake. In any of the embodiments described herein, the subject can be obese. In any of the embodiments described herein, the subject can have excessive weight. In any of the embodiments described herein, the subject can have elevated BMI. In any of the embodiments described herein, the subject can have elevated body fat mass, percentage, or volume. In any of the embodiments described herein, the subject can have excessive food intake.

Symptoms of obesity include, but are not limited to, excess body fat accumulation (particularly around the waist), breathlessness, increased sweating, snoring, inability to cope with sudden physical activity, feeling extra tired every day, back and joint pains, skin problems (from moisture accumulating in the folds of skin).

Methods of treating a subject having obesity, the methods comprising administering a GPR75 inhibitor to the subject, are provided. Also disclosed are methods of treating a subject having excessive weight, the methods comprising administering a GPR75 inhibitor to the subject. The present disclosure also provides methods of treating a subject having elevated BMI, the methods comprising administering a GPR75 inhibitor to the subject. Methods of treating a subject having elevated body fat mass, percentage, or volume, the methods comprising administering a GPR75 inhibitor to the subject are also described. Finally, treatment of a subject having excessive food intake, comprising administering a GPR75 inhibitor to the subject are also disclosed.

The present disclosure also provides methods of treating a subject to prevent weight gain or to maintain weight loss, the method comprising administering a GPR75 inhibitor to the subject.

In certain embodiments, methods of treating a metabolic disease or disorder in a subject, comprising administering an effective amount of GPR75 inhibitor to the subject are provided.

As used herein, the term “metabolic disease” or “metabolic disorder” is also called a metabolic syndrome and refers to a set of abnormal states such as an increase in body fat, an increase in blood pressure, an increase in blood sugar, and abnormal lipids in blood, which increase the risk of cerebral cardiovascular diseases and diabetes mellitus. The metabolic disease is not a single disease, but a comprehensive disease caused by genetic predisposition and environmental factors, and in the present invention, may be selected from the group consisting of obesity, diabetes mellitus, dyslipidemia, insulin resistance, hepatic steatosis, hypercholesterolemia, and non-alcoholic fatty liver disease, and may be more preferably obesity or diabetes mellitus, but is not limited thereto.

As used herein, the term “diabetes mellitus”, as a type of metabolic disease such as an insufficient amount of insulin secreted or an absence of normal function, is characterized by high blood sugar with high blood glucose concentration and causes various symptoms and signs due to hyperglycemia and glucose release from urine. Diabetes mellitus includes type 1 diabetes mellitus which occurs when insulin is not secreted largely due to the destruction of pancreatic beta cells, and type 2 diabetes mellitus which is caused by insufficient insulin secretion in the body or insulin resistance in which cells do not respond to insulin. In the present invention, diabetes mellitus includes both type 1 diabetes mellitus and type 2 diabetes mellitus. In certain embodiments, the method further comprises administering a second therapeutic agent that treats or inhibits obesity. Nonlimiting examples of therapeutic agents that treat or inhibit obesity and/or increased BMI include, but are not limited to, GLP-1R agonists, melanocortin 4 receptor (MC4R) agonists, sibutramine, orlistat, phentermine, lorcaserin, naltrexone, liraglutide, diethylpropion, bupropion, metformin, pramlintide, topiramate, and zonisamide, or any combination thereof.

Administration of the therapeutic agents that treat or inhibit obesity and/or GPR75 inhibitors can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period such as, for example, 6 months, 1 year, or more.

Administration of the therapeutic agents that treat or inhibit obesity and/or GPR75 inhibitors can occur by any suitable route including, but not limited to, parenteral, intravenous, oral, lingual, buccal, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The compounds can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients. See, e.g., Remington's Pharmaceutical Sciences, latest edition, by E. W. Martin Mack Pub. Co., Easton, PA, which discloses typical carriers and conventional methods of preparing pharmaceutical compositions that can be used in conjunction with the preparation of formulations of the compounds described herein and which is incorporated by reference herein. These most typically would be standard carriers for administration of compositions to humans. In one aspect, humans and non-humans, including solutions such as sterile water, saline, and buffered solutions at physiological pH. In preferred embodiments, the compositions include a HEPES buffer. Other compounds will be administered according to standard procedures used by those skilled in the art.

These compositions can take the form of solutions, suspensions, emulsion, tablets, pills, capsules, powders, sustained-release formulations, and the like.

In some embodiments, the therapeutic agents that treat or inhibit obesity and/or GPR75 inhibitors (such as any of the peptide ligands disclosed herein) are administered intrathecally (i.e., introduction into the subarachnoid space of the spinal cord or into the spinal canal so that the therapeutic agent can reach the cerebrospinal fluid of a subject, or introduction into the anatomic space or potential space inside a sheath, including, by way of non-limiting examples, the arachnoid membrane of the brain or spinal cord). In some embodiments, intrathecal administration results in the therapeutic agent acting on, without limitation, the cortex, the cerebellum, the striatum, the cervical spine, the lumbar spine, or the thoracic spine. Therapeutic agents administered intrathecally may ultimately act on targets throughout the entire central nervous system. In some embodiments, the intrathecal administration is into the cisterna magna or by the lumbar area or region. In some embodiments, the intrathecal administration into the lumbar area or region results in delivery of the therapeutic agent to the distal spinal canal.

Exemplary methods for intrathecal administration are described in, for example, Lazorthes et al., Advances in Drug Delivery Systems and Applications in Neurosurgery, 143-192. In some embodiments, the intrathecal administration is by injection, by bolus injection, by a catheter, or by a pump. In some embodiments, the intrathecal administration is by lumber puncture. In some embodiments, the pump is an osmotic pump. In some embodiments, the pump is implanted into subarachnoid space of the spinal canal, below the skin of the abdomen, or behind the chest wall. In some embodiments, the intrathecal administration is by an intrathecal delivery system for a therapeutic substance including a reservoir containing a volume of the therapeutic agent and a pump configured to deliver at least a portion of the therapeutic substance contained in the reservoir. In some embodiments, intrathecal administration is through intermittent or continuous access to an implanted intrathecal drug delivery device (IDDD). In some embodiments, the therapeutic substance is an inhibitory nucleic acid molecule. In some embodiments, the amount of the nucleic acid molecule or peptide molecule administered intrathecally ranges from about 10 μg to about 2 mg, from about 50 μg to about 1500 μg, or from about 100 μg to about 1000 μg. In some embodiments, the therapeutic agent is disposed within a pharmaceutical composition. In some embodiments, the pharmaceutical composition does not comprise a preservative.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated rangc.

### Parenteral Formulations

The peptides described herein can be formulated for parenteral administration. For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, intralingually, subconjunctivally, intravesicularly, intrapericardially, intraumbilically, by injection, and by infusion.

Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsosomes.

If for intravenous administration, the compositions are packaged in solutions of sterile isotonic aqueous buffer. In certain embodiments, the compositions are packaged in solutions containing a HEPES buffer. Where necessary, the composition may also include a solubilizing agent. The components of the composition are supplied either separately or mixed in unit dosage form, for example, as a dry lyophilized powder or concentrated solution in a hermetically sealed container such as an ampoule or sachet indicating the amount of active agent. If the composition is to be administered by infusion, it can be dispensed with an infusion bottle containing sterile pharmaceutical grade water or saline. Where the composition is administered by injection, an ampoule of sterile water or saline can be provided so that the ingredients may be mixed prior to injection.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, using a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric, or nonionic surface-active agents. Suitable anionic surfactants include, but are not limited to, those containing carboxylate, sulfonate and sulfate ions. Examples of anionic surfactants include sodium, potassium, ammonium of long chain alkyl sulfonates, and alkyl aryl sulfonates such as sodium dodecylbenzene sulfonate; dialkyl sodium sulfosuccinates, such as sodium dodecylbenzene sulfonate: dialkyl sodium sulfosuccinates, such as sodium bis-(2-ethylthioxyl)-sulfosuccinate; and alkyl sulfates, such as sodium lauryl sulfate. Cationic surfactants include, but are not limited to, quaternary ammonium compounds, such as benzalkonium chloride, benzethonium chloride, cetrimonium bromide, stearyl dimethylbenzyl ammonium chloride, polyoxyethylene, and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine, and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers. In certain embodiments, the formulation includes a HEPES buffer.

Water-soluble polymers are often used in formulations for parenteral administration. Suitable water-soluble polymers include, but are not limited to, polyvinylpyrrolidonc, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

The parenteral formulations described herein can be formulated for controlled release including immediate release, delayed release, extended release, pulsatile release, and combinations thereof.

### Nano- and Microparticles

For parenteral administration, the one or more compounds, and optional one or more additional active agents, can be incorporated into microparticles, nanoparticles, or combinations thereof that provide controlled release of the compounds and/or one or more additional active agents. In forms wherein the formulations contain two or more peptides, the peptides can be formulated for the same type of controlled release (e.g., delayed, extended, immediate, or pulsatile) or the peptides can be independently formulated for different types of release (e.g., immediate and delayed, immediate and extended, delayed and extended, delayed and pulsatile, etc.).

For example, the compounds and/or one or more additional active agents can be incorporated into polymeric microparticles, which provide controlled release of the peptide(s). Release of the peptide(s) is controlled by diffusion of the protein(s) out of the microparticles and/or degradation of the polymeric particles by hydrolysis and/or enzymatic degradation. Suitable polymers include ethylcellulose and other natural or synthetic cellulose derivatives.

Polymers, which are slowly soluble and form a gel in an aqueous environment, such as hydroxypropyl methylcellulose or polyethylene oxide, can also be suitable as materials for protein containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB), and copolymers thereof, poly-4-hydroxybutyrate (P4HB) and copolymers thereof, polycaprolactone and copolymers thereof, and combinations thereof.

Alternatively, the protein(s) can be incorporated into microparticles prepared from materials which are insoluble in aqueous solution or slowly soluble in aqueous solution but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances, and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and triglycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins, and candelilla wax. As used herein, a wax-like material is defined as any material, which is normally solid at room temperature and has a melting point of from about 30 to 300° C.

In some cases, it may be desirable to alter the rate of water penetration into the microparticles. To this end, rate-controlling (wicking) agents can be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (e.g., lecithin) may be added to facilitate the degradation of such microparticles.

Proteins, which are water insoluble, such as zein, can also be used as materials for the formation of protein containing microparticles. Additionally, proteins, polysaccharides and combinations thereof, which are water-soluble, can be formulated with peptide into microparticles and subsequently cross-linked to form an insoluble network. For example, cyclodextrins can be complexed with individual drug molecules and subsequently cross-linked.

### Method of Making Nano- and Microparticles

Encapsulation or incorporation of drug into carrier materials to produce drug-containing microparticles can be achieved through known pharmaceutical formulation techniques. In the case of formulation in fats, waxes, or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling, or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. These processes are known in the art.

For some carrier materials it may be desirable to use a solvent evaporation technique to produce drug-containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some forms, drug in a particulate form is homogeneously dispersed in a water-insoluble or slowly water-soluble material. To minimize the size of the drug particles within the composition, the drug powder itself may be milled to generate fine particles prior to formulation. The process of jet milling, known in the pharmaceutical art, can be used for this purpose. In some forms, drug in a particulate form is homogeneously dispersed in a wax or wax-like substance by heating the wax or wax-like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation, and gamma irradiation.

To produce a coating layer of cross-linked protein surrounding drug containing microparticles or drug particles, a water-soluble protein can be spray coated onto the microparticles and subsequently cross-linked by the one of the methods described above. Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose, and guar gum are subject to cross-linking in the presence of multivalent cations. Complexes between oppositely charged polysaccharides can also be formed; pectin and chitosan, for example, can be complexed via electrostatic interactions.

### Injectable/Implantable Formulations

The compounds described herein can be incorporated into injectable/implantable solid or semi-solid implants, such as polymeric implants. In some forms, the compounds are incorporated into a polymer that is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication requires polymers having a melting point that is below the temperature at which the substance to be delivered and polymer degrade or become reactive. The device can also be prepared by solvent casting where the polymer is dissolved in a solvent and the drug dissolved or dispersed in the polymer solution and the solvent is then evaporated. Solvent processes require that the polymer be soluble in organic solvents. Another method is compression molding of a mixed powder of the polymer and the drug or polymer particles loaded with the active agent.

Alternatively, the compounds can be incorporated into a polymer matrix and molded, compressed, or extruded into a device that is a solid at room temperature. For example, the compounds can be incorporated into a biodegradable polymer, such as polyanhydrides, polyhydroalkanoic acids (PHAs). PLA, PGA, PLGA, polycaprolactone, polyesters, polyamides, polyorthoesters, polyphosphazenes, proteins and polysaccharides such as collagen, hyaluronic acid, albumin and gelatin, and combinations thereof and compressed into solid device, such as disks, or extruded into a device, such as rods.

The release of the one or more compounds from the implant can be varied by selection of the polymer, the molecular weight of the polymer, and/or modification of the polymer to increase degradation, such as the formation of pores and/or incorporation of hydrolyzable linkages. Methods for modifying the properties of biodegradable polymers to vary the release profile of the compounds from the implant are well known in the art.

### Enteral/Oral/Lingual Formulations

Oral/lingual formulations can include standard carriers such as pharmaceutical grades of mannitol, lactose, sodium saccharine, starch, magnesium stearate, cellulose, magnesium carbonate, etc. Such compositions will contain a therapeutically effective amount of the compound and/or antibiotic together with a suitable amount of carrier to provide the proper form to the patient based on the mode of administration to be used.

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition, which may include plasticizers, pigments, colorants, stabilizing agents, and glidants.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

“Diluents”, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

“Binders” are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

“Lubricants” are used to facilitate tablet manufacture. Examples of suitable lubricants include, but are not limited to, magnesium stearate, calcium stearate, stearic acid, glycerol behenate, polyethylene glycol, talc, and mineral oil.

“Disintegrants” are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross-linked polymers, such as cross-linked PVP (Polyplasdone® XL from GAF Chemical Corp).

“Stabilizers” are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

Oral dosage forms, such as capsules, tablets, solutions, and suspensions, can for formulated for controlled release. For example, the one or more compounds and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation into the finished dosage form.

In another form, the one or more compounds and optional one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another form, the one or more compounds, and optional one or more additional active agents are formulated into a sold oral dosage form, such as a tablet or capsule, and the solid dosage form is coated with one or more controlled release coatings, such as a delayed release coatings or extended-release coatings. The coating or coatings may also contain the compounds and/or additional active agents.

The materials and methods below are provided to facilitate the practice of the present invention.

### Peptide Synthesis and Purification

Solid-Phase Peptide Synthesis was performed on ProTide Rink amide resin (CEM Corporation cat #R002) using a microwave-assisted CEM Liberty Blue peptide synthesizer (Matthews, NC). Fmoc-protected amino acids were coupled to the resin using 0.25 M Oxyma Pure (CEM Corporation cat #S001) and 0.125 M N,N′-diisopropylcarbodiimide (Sigma-Aldrich cat #D125407) as the activator and activator base, respectively. Fmoc was removed between couplings with 20% Piperidine (Sigma-Aldrich cat #8.22299.0500). Global deprotection and cleavage of the peptides from the solid-support resin achieved using a CEM Razor instrument over a 40-minute incubation period at 40° C. in a mixture of 95% TFA (Sigma-Aldrich cat #8.08260.2501), 2.5% TIPS (Sigma-Aldrich cat #233781), and 2.5% water. Peptides were purified on an Agilent 1200 series High-Performance Liquid Chromatography (HPLC) instrument (10-75% HPLC-grade acetonitrile (VWR cat #BDH83639.400) for 20 minutes at 2 mL/min flow rate using an Agilent Zorbax C18 column (5 μm, 9.4×250 mm) tracked at 280 nm.

### Binding Analysis of Peptides at GPR75

SU75-36 binding at the human GPR75 (372-540 aa region; available on the world wide web at antibodies-online.com cat #ABIN5709609) was measured via a Nicoya Open SPR instrument using His-tagged GPR75 bound to an NTA-coated gold sensor (Nicoya cat #SEN-AU-100-10-NTA, range=4.08-204 μM) using HBSS (in-house) at 20 μL/min flow rate. Data was fit via a global, one-to-one model using Nicoya OpenSPR software.

Binding Analysis of Peptides at hGLP-1R

SU75-36, SU75-37, Ex-4, and ODN binding at the human GLP-1R (21-139aa region; available on the world wide web at rndsystems.com cat #10956-GL) was measured via a Nicoya Open SPR instrument using His-tagged hGLP-1R bound to an NTA-coated gold sensor (Nicoya cat #SEN-AU-100-10-NTA) using HBSS (in-house) at 20 μL/min flow rate. Data was fit via a global, one-to-one model using Nicoya OpenSPR software. In-house Ex-4 and ODN were used as the positive and negative controls, respectively.

### Circular Dichroism (CD) Spectroscopy

Peptides were prepared in 0.5% saline (pH 7.4) at 40 UM for folded-state analysis on a Chirascan VX (Applied Photophysics, Leatherhead, Surrey, England) spectropolarimeter. Samples were run as duplicate data sets, each as quartet replicates, using a 1 cm quartz cell, 200-260 nm measurement range, 100 nm/min scanning speed, 1 nm bandwidth, 4 second response time, and 1 nm data pitch. The averaged data output was converted from Δε (M⁻¹ cm⁻¹) to molar ellipticity to then obtain their corresponding percent helicity values.

### Animals

Adult male Sprague-Dawley rats (Charles River) were individually housed under a 12 h-light: 12 h-dark cycle in a temperature and humidity-controlled satellite vivarium and had ad libitum access to water and chow (5001, LabDiet) or a 60% high fat diet (HFD): D12492, Research Diets) and when applicable had ad libitum access to kaolin pellets (K50001, Research Diets). Rats were exposed to kaolin for at least 5 days prior to measuring kaolin consumption in pica testing. Except for studies conducted in the bioDAQ, for all feeding studies rats were housed in hanging wire cages to allow for accurate measurement of food spillage. Experiments were conducted under the National Institutes for Health Guide for the Care and Use of Laboratory Animals and all procedures were approved by the Institutional Animal Care and Use Committee at the University of Pennsylvania.

### Surgeries

For cannula implantation, rats were anesthetized by intraperitoneal injection of a mixture containing ketamine (90 mg/kg, Butler Animal Health Supply), xylazine (2.7 mg/kg, Anased), and acepromazine (0.64 mg/kg, Butler Animal Health Supply) (KAX) and then placed into a stereotaxic apparatus. Each rat was stereotaxically implanted with a guide cannula (26-ga, Plastics One) aimed at the fourth ventricle (guide cannula coordinates: on midline. 2.5 mm anterior to occipital suture, 5.2 mm ventral to skull; internal cannula aimed 7.2 mm ventral to skull) or the lateral ventricle (guide cannula coordinates: 1.5 mm lateral to midline, 0.9 mm posterior to bregma. 1.8 mm ventral to skull; internal cannula aimed 3.8 mm ventral to skull). For all cannulas, dummies (no projection beyond guide) were inserted in the guide cannula and left until infusions were performed. For all surgeries, rats received post-operative temperature support and analgesia was provided immediately following surgery and for two post-operative days (2 mg/kg meloxicam).

### Food and Kaolin Intake Studies

For all studies measuring food intake following drug treatment, central injections were given at a volume of 2 μL using a Hamilton syringe terminating in an injector tip extending 2.0 mm beyond the guide cannula. For acute treatment days, rats were food deprived for 2 hours before the dark cycle and injections were done immediately prior to the dark cycle onset. Food and kaolin intake was measured 1, 3, 6, and 24 hours after injections were completed and food crumbs were weighed and accounted for between each timepoint. Body weight was measured during injections and 24 hours after. Injection treatments were organized in a counterbalanced, within-subjects design and separated by ≥72 h.

### Drugs

All drugs (SUODN36 (SU75-36) and SUODN37 (SU75-37)) used in these studies were synthesized by the Doyle lab at Syracuse University. In all cases, drugs were dissolved in artificial cerebrospinal fluid (aCSF, Harvard Apparatus). The sequence for SU75-36 and SU75-37 are provided in Table 1.

The following examples are provided to illustrate certain embodiments of the invention. They are not intended to limit the invention in any way.

### Example I: Peptide Ligands that Bind GPR75

Herein, we describe a novel non-naturally occurring peptide ligand for the highly sought-after orphan GPR75. GPR75 is a major drug discovery goal of the pharmaceutical industry given the association of GPR75 with obesity and metabolic diseases.

Non-naturally occurring peptides were produced using the methods described above. Table 4 provides the sequences of two novel, non-naturally occurring peptide sequences for antagonizing GPR75.

TABLE 4
Peptide Sequences. Peptides have been synthesized, confirmed, and purified
prior to testing. Lowercase letter indicates D-amino acid. Peptides are
C-terminally amidated.

| Peptide | Sequence | SEQ ID NO |
|---|---|---|
| SU75-36 | HsQGTFTSDLSKYLEEEVREFIWLKNGGPSDVNTDRPGLLDLK-NH₂ | 1 |
| SU75-37 | TFTSDLSKYLEEEVREFIWLKNGGPSDVNTDRPGLLDLK-NH₂ | 2 |

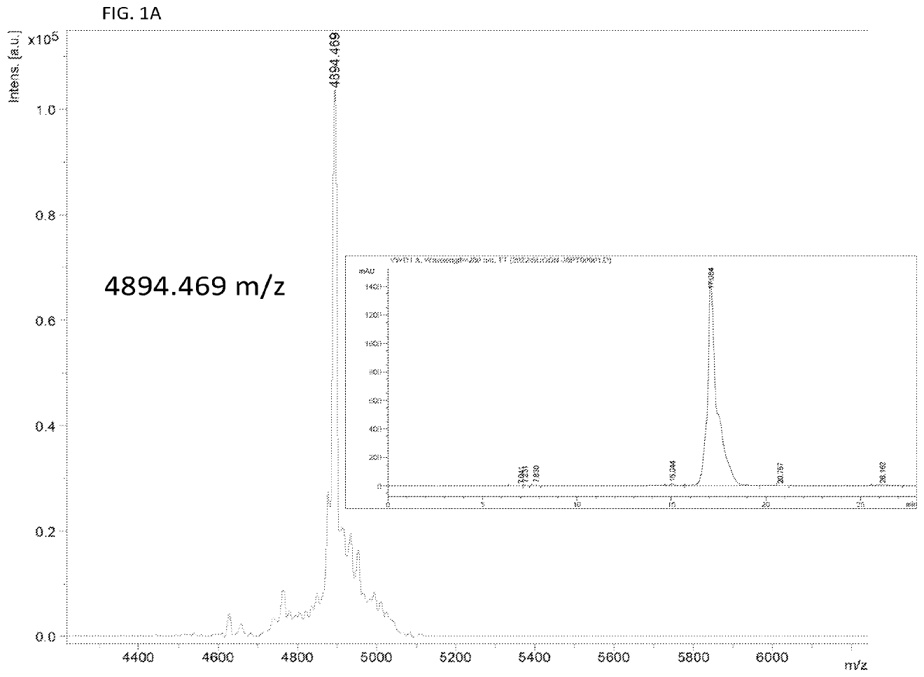

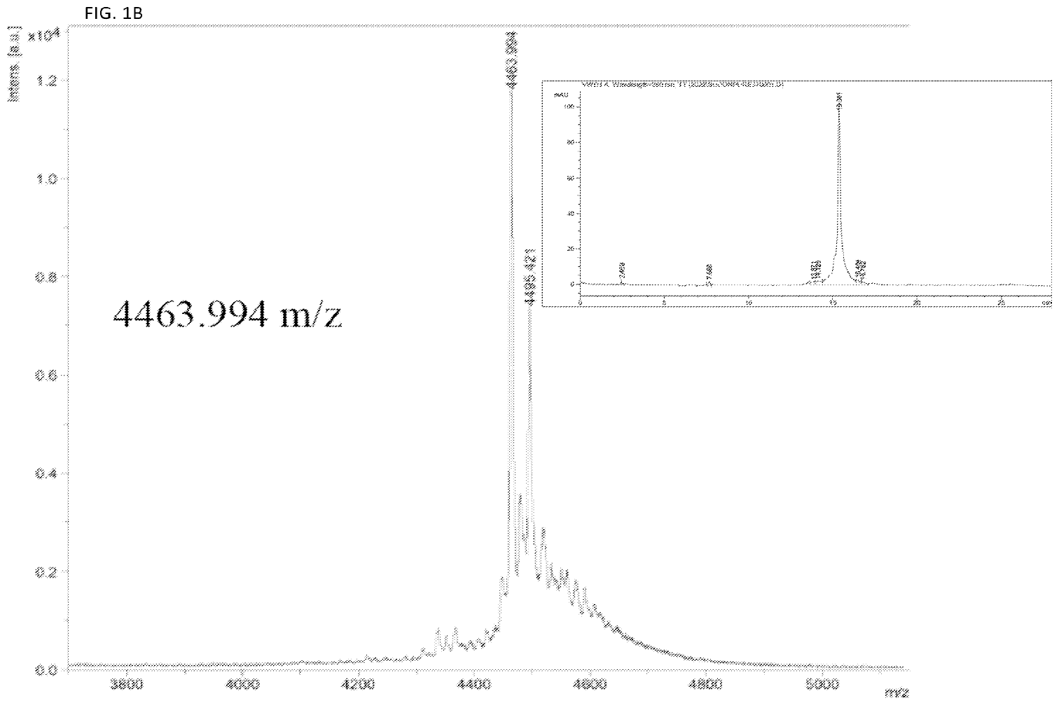

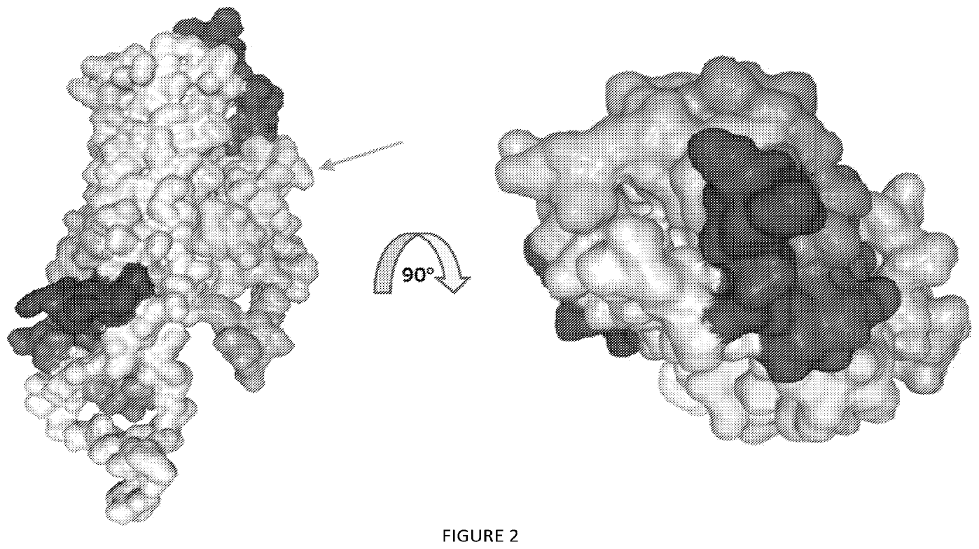

Peptide synthesis and purity were confirmed using High-Performance Liquid Chromatography (HPLC). FIG. 1A shows SU75-36 synthesis at 100% purity while FIG. 1B shows SU75-37 synthesis at 98.3% purity.

The binding of SU75-36 to GPR75 was then modeled using HPEPDOCK. Specifically, blind SU75-36/GPR75 receptor in silico docking using HPEPDOCK shows a Docking score of 0.884. Successful administration of SU75-37, as shown herein, indicates that SU75-37 binds the GPR75 receptor similarly.

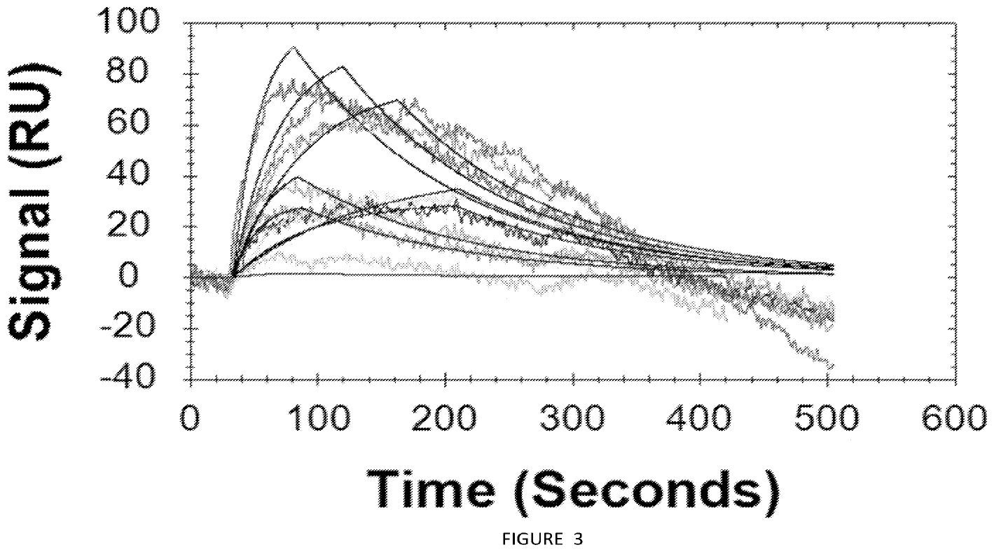

The binding of SU75-36 was further analyzed using a Surface Plasmon Resonce (SPR) assay which tracks binding of SU75-36 to GPR75 over time. The assay indicated that SU75-36 binds to GPR75 with a K_{D} of 7.76 μM. (FIG. 3) This assay was further analyzed using Nicoya OpenSPR software which showed the best fit parameters using a one-to-one model. The data obtained from this software is present in Tables 5 and 6. K_{D} was observed to be 7.73×10⁻⁶ M (7.76 μM).

TABLE 5
Evaluation type: OneToOne

| Curve name | Bmax ([Signal (RU)]) | ka (1/(M*s)) | kd (1/s) | KD (M) | BI ([Signal (RU)]) |
|---|---|---|---|---|---|
| Drug 36_49.6 μM_7962s fitted | 69.54 | 9.56e2 | 7.42e−3 | 7.76e−6 | 0.00 |
| Drug 36_24.8 μM_8902.74s fitted | 69.54 | 9.56e2 | 7.42e−3 | 7.76e−6 | 0.00 |
| Drug 36_102 μM_9594.85s fitted | 69.54 | 9.56e2 | 7.42e−3 | 7.76e−6 | 0.00 |
| Drug 36_10.2 μM_10677.83s fitted | 69.54 | 9 56e2 | 7.42e−3 | 7.76e−6 | 0.00 |
| Drug 36_63 μM_12735 07s fitted | 69.54 | 9.56e2 | 7.42e−3 | 7.76e−6 | 0.00 |
| Drug 36_102 μM_13348.46s fitted | 69.54 | 9.56e2 | 7.42e−3 | 7.76e−6 | 0.00 |

TABLE 6

| | Chi2 | U-value: ka |
|---|---|---|
| Curve name | ([Signal (RU)]\hat{ }2) | (%) |
| Drug 36_49.6 μM_7962s fitted | 207.75 | 13.90 |
| Drug 36_24.8 μM 8902.74s fitted | 207.75 | 13.90 |
| Drug 36_102 μM_9594.85s fitted | 207.75 | 13.90 |
| Drug 36_10.2 μM_10677.88s fitted | 207.75 | 13.90 |
| Drug 36_68 μM_12735.07s fitted | 207.75 | 13.90 |
| Drug 36_102 μM_13348 46s fitted | 207.75 | 13.90 |

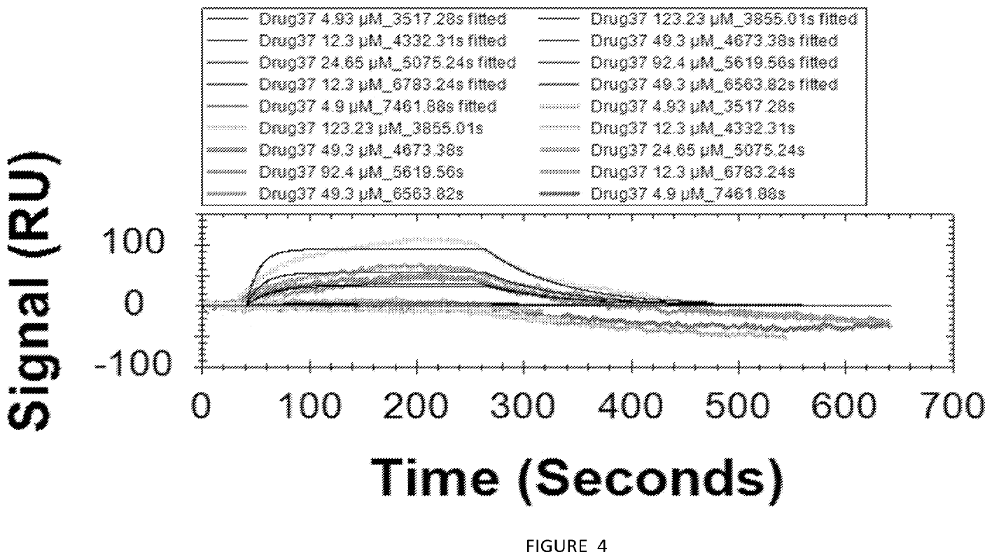

The SPR assay was duplicated to determine the binding of SU75-37 to GPR75 over time. The assay indicated that SU75-37 binds to GPR75 with a K_{D} of 23.8 μM. (FIG. 4) As with SU75-36, this assay was further analyzed using Nicoya OpenSPR software. The data obtained from this software is present in Table 7. K_{D} was observed to be 2.38×10⁻⁵ M (23.8 UM).

TABLE 7

| Curve name | Bmax ([Signal (RU)]) | ka (1/(M*s)) | kd (1/s) | KD (M) | BI ([Signal (RU)]) |
|---|---|---|---|---|---|
| Drug37 4.93 μM_3517.28s fitted | 3.57 | 5.92e2 | 1.41e−2 | 2.38e−5 | 0.10 |
| Drug37 123.23 μM_3855.01s fitted | 110.32 | 5.92e2 | 1.41e−2 | 2.38e−5 | 0.10 |
| Drug37 12.3 μM_4332.31s fitted | 9.39 | 5.92e2 | 1.41e−2 | 2.38e−5 | 0.10 |
| Drug37 49.3 μM_4673.38s fitted | 51.69 | 5.92e2 | 1.41e−2 | 2.38e−5 | 0.10 |
| Drug37 24.65 μM_5075.24s fitted | 8.98 | 5.92e2 | 1.41e−2 | 2.38e−5 | 0.10 |
| Drug37 92.4 μM_5619.56s fitted | 67.67 | 5.92e2 | 1.41e−2 | 2.38e−5 | 0.10 |
| Drug37 12.3 μM_6783.24s fitted | 11.98 | 5.92e2 | 1.41e−2 | 2.38e−5 | 0.10 |
| Drug37 49.3 μM_6563.82s fitted | 47.66 | 5.92e2 | 1.41e−2 | 2.38e−5 | 0.10 |
| Drug37 4.9 μM_7461.88s fitted | 3.80 | 5.92e2 | 1.41e−2 | 2.38e−5 | 0.10 |

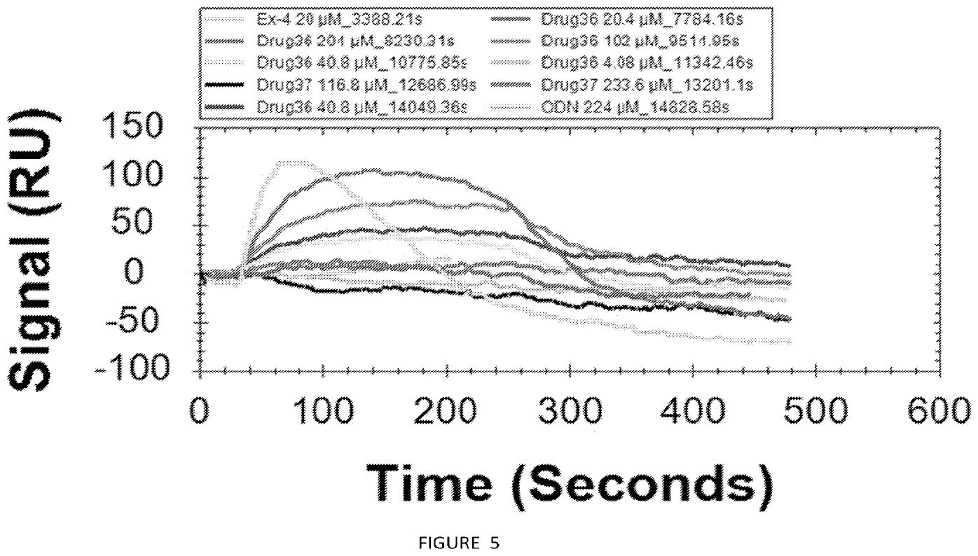

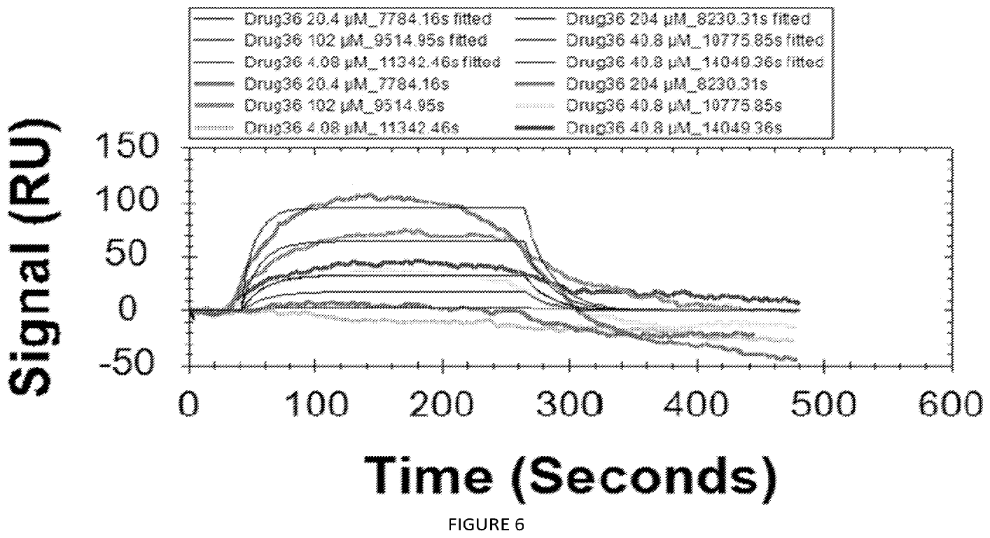

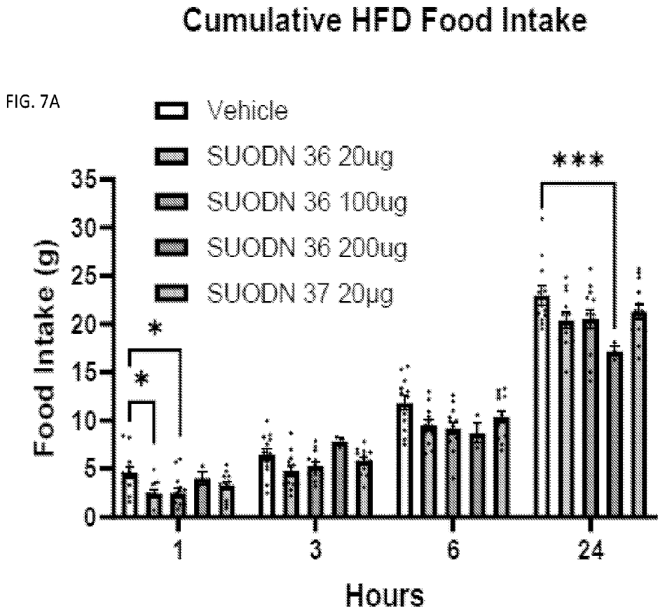

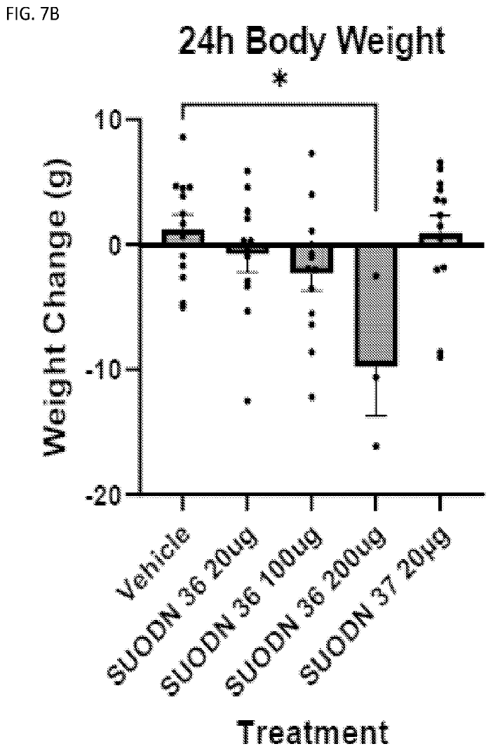

To confirm the specificity of the peptide ligand binding, the SPR assay was duplicated for both SU75-36 and SU75-37 to determine binding at hGLP-1R with Ex-4 and ODN used as a positive and negative control respectively. SU75-37 did not bind to the hGLP-1R receptor at all. (FIG. 5). Although SU75-36 showed the ability to bind the hGLP-1R receptor, it had a K_{D} of 182 μM which is indicative of poor binding at this receptor. (FIG. 7)

Circular dichroism (CD) spectroscopy was used to analyze the folded-states of both SU75-36 and SU75-37. The peptides each showed spectra indicative of a typical α-helix with percent helicity values greater than 20%. (FIG. 9)

### Example II: Peptide Ligands Administration to Rats

Herein, we describe administration of novel non-naturally occurring peptide ligand as GPR75 inhibitor for the treatment of obesity.

Diet induced obese (DIO) rats (n=26) were administered 20 μg SU75-36, 100 μg SU75-36, 200 μg SU75-36, or 20 μg SU75-37 by intracerebroventricular injection to the 4th ventricle and observed over 24 hours. One group of rats was administered a vehicle without a GPR75 inhibitor as a negative control. All rats were given ad libitum access to water and 60% high fat diet (HFD). The food intake of each rat was observed 1, 3, 6, and 24 hours after administration of the GPR75 inhibitor. (FIG. 7A) Rats in each group that was administered an GPR75 inhibitor showed a significant decrease in food intake when compared to the control. Rats that were administered 200 μg SU75-36 showed a further decrease when compared to the any of the other groups.

Rats were weighed at the beginning of the experiment and 24 hours after administration of the GPR75 inhibitor. The weight change of each rat after 24 hours was then calculated. Rats in the control group showed a slight increase in weight after 24 hours. Rats administered any amount of SU75-36 showed a significant decrease in weight after 24 hours. (FIG. 7B).

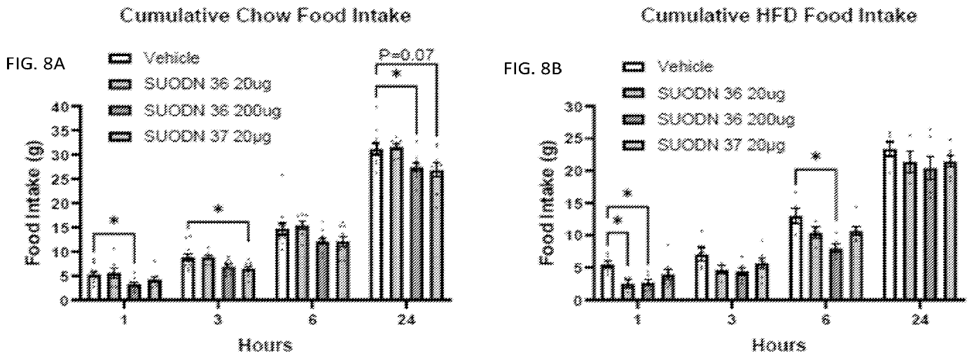

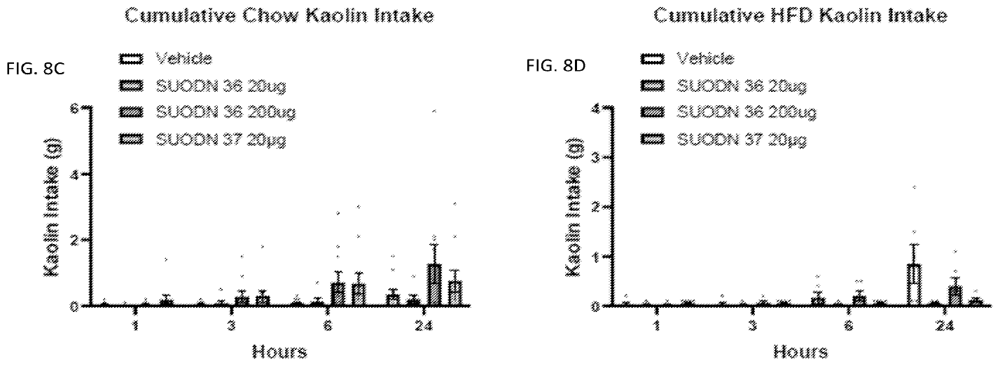

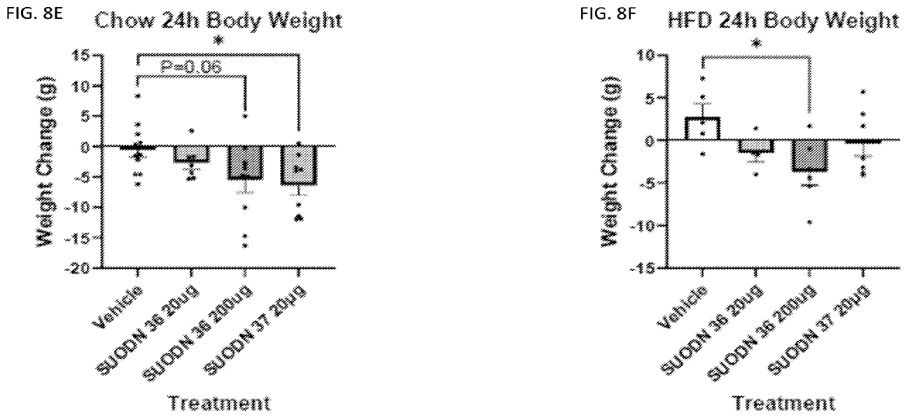

In a separate experiment, chow maintained (n=10) and diet-induced obese (DIO) rats (n=12) were administered 20 μg SU75-36, 200 μg SU75-36, or 20 μg SU75-37 by intracerebroventricular injection to the lateral ventricle and observed over 24 hours. One group of rats was administered a vehicle without a GPR75 inhibitor as a negative control. All rats were given ad libitum access to access to water, kaolin pellets to assess pica behavior, and food, either a standard chow diet or a 60% high fat diet (HFD). The food intake of chow (FIG. 8A) or HFD (FIG. 8B), and kaolin take of chow-maintained rats (FIG. 8C) and HFD-maintained rats (FIG. 8D) of each rat was observed 1, 3, 6, and 24 hours after administration of the GPR75 inhibitor. Rats in each group that was administered an GPR75 inhibitor showed a significant decrease in food intake when compared to the control. Rats that were administered 200 μg SU75-36 showed a further decrease when compared to the any of the other groups. Additionally, the data showed an absence of kaolin intake, an established model of malaise and nausea. This indicates that antagonizing GPR75 with these novel antagonists suppresses energy balance without producing nausea/malaise.

Rats were weighed at the beginning of the experiment and 24 hours after administration of the GPR75 inhibitor. The weight change of each rat after 24 hours was then calculated. Rats in the control group fed HFD chow showed a slight increase in weight after 24 hours. Rats administered any dosage of SU75-36 or SU75-37 showed a significant decrease in weight after 24 hours when fed chow or HFD chow. (FIGS. 8E, 8F).

### Example III: Administration of Peptide Ligands to Rats in a HEPES Buffer

Next, the appetite suppressive effect of the novel non-naturally occurring peptide ligands in different solutions, including a HEPES buffer and DMSO, was analyzed.

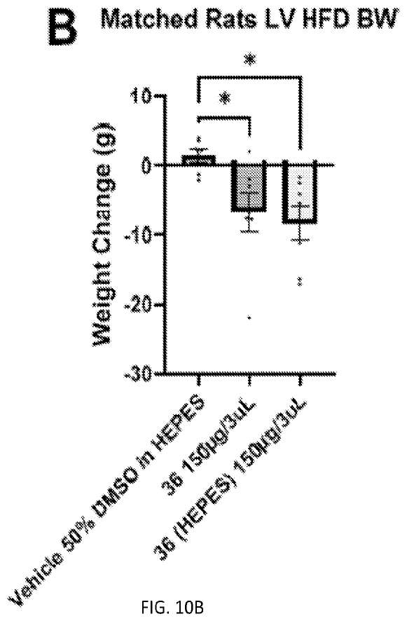

Diet induced obese (DIO) rats (n=4) were administered 150 μg/3 μL SU75-36 in a vehicle or in a HEPES buffer by lateral intraventricular injection and observed over 24 hours. One group of rats was administered a Vehicle of 50% DMSO in HEPES as a negative control. All rats were given ad libitum access to water and 60% HFD. The food intake of each rat was observed at 1, 3, 6, and 24 hours after administration. (FIG. 10A). Rats in each group that was administered SU75-36 showed a significant decrease in food intake when compared to the control. Rats that were administered the SU75-36 in the HEPES buffer showed a further decrease when compared to the any of the other groups.

Rats were weighed at the beginning of the experiment and 24 hours after administration. The weight change of each rat after 24 hours was then calculated. Rats in the control group showed a slight increase in weight after 24 hours. Rats administered SU75-36 showed a significant decrease in weight after 24 hours. Rats administered SU75-36 with the HEPES buffer showed a further decrease when compared to any other of the groups. (FIG. 10B).

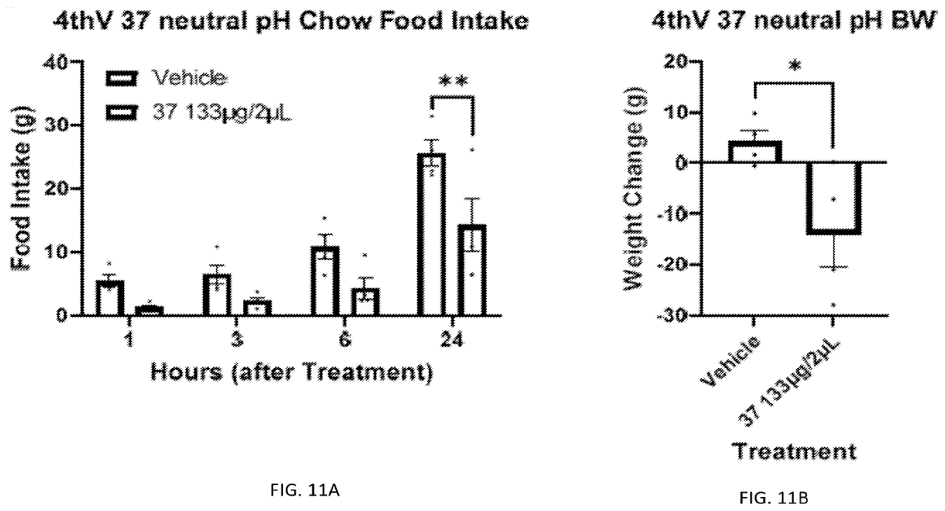

In a separate experiment, diet induced obese (DIO) rats (n=4) were administered 133 μg/2 μL SU75-37 in a HEPES buffer by lateral intraventricular injection and observed over 24 hours. One group of rats was administered a vehicle without a GPR75 inhibitor as a negative control. All rats were given ad libitum access to water and a 60% high fat diet (HFD). SU75-37 was optimized using an acetate salt precipitation rather than a TFA salt precipitation to maintain a neutral >4.5 pH in HEPES buffer solution. The food intake of each rat was observed 1, 3, 6, and 24 hours after administration. (FIG. 11A) Rats that were administered SU75-37 in a HEPES buffer a significant decrease in food intake when compared to the control.

Rats were weighed at the beginning of the experiment and 24 hours after administration. The weight change of each rat after 24 hours was then calculated. Rats in the control group showed a slight increase in weight after 24 hours. Rats administered SU75-37 in a HEPES buffer showed a significant decrease in weight after 24 hours. (FIG. 11B).

### Example IV: Peripheral Administration of Peptide Ligands to Rats

Next, the appetite suppressive effect of the novel non-naturally occurring peptide ligands when administered peripherally was analyzed.

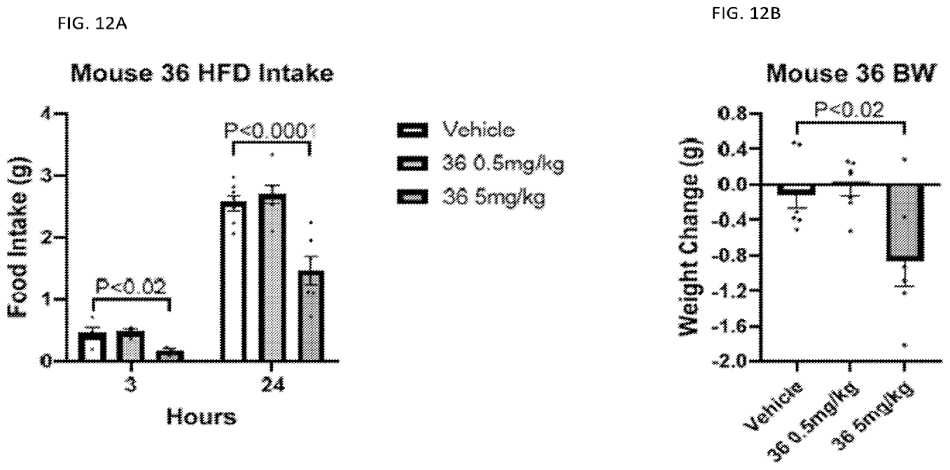

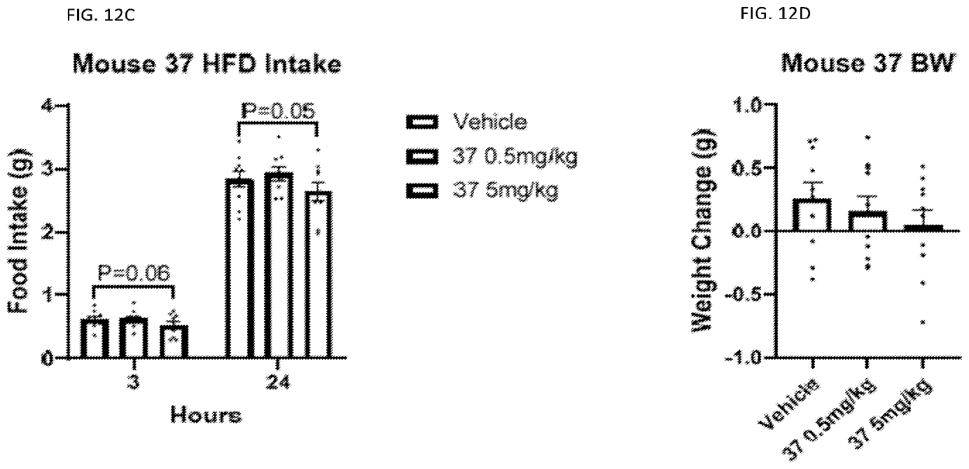

DIO mice were administered 0.5 mg/kg or 5.0 mg/kg of either SU75-36 or SU75-37 in a vehicle by intra-peripheral injection and observed over 24 hours. One group of mice was administered a vehicle as a negative control. All mice were given ad libitum access to water and a 60% HFD. The food intake of each mouse was observed at 3 and 24 hours after administration. (FIGS. 12A and 12C). Mice that were administered SU75-36 or SU75-37 at 5 mg/kg showed a significant decrease in food intake when compared to the control.

Mice were weighed at the beginning of the experiment and 24 hours after administration. The weight change of each mouse after 24 hours was then calculated. Mice in the control group showed an increase or a slight decrease in weight after 24 hours. Mice administered SU75-36 showed a significant decrease in weight after 24 hours. (FIGS. 12B and 12D)

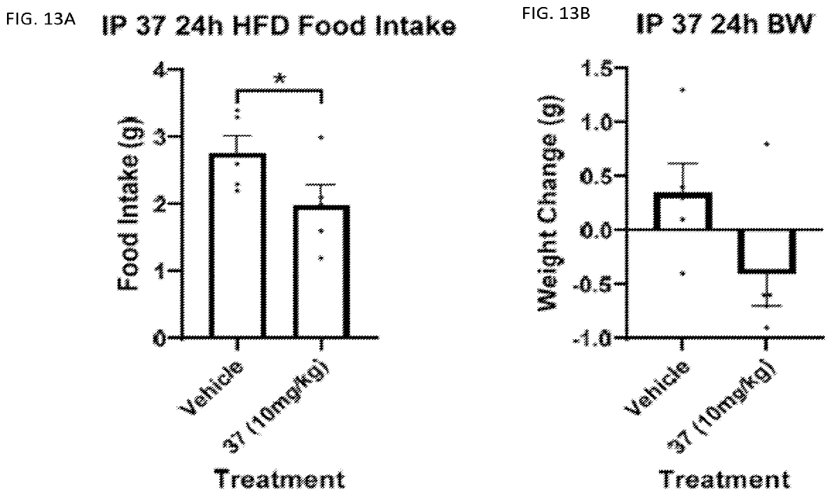

This experiment was then repeated for SU75-37 at a higher dose (10 mg/kg). At this higher dose, the mice showed a significant decrease in food intake when compared to the control. (FIG. 13A). The mice also showed a significant decrease in weight after 24 hours (FIG. 13B).

### Example V: Administration of Peptide Ligands to Human Patients

The information herein above can be applied clinically to patients for therapeutic intervention. A preferred embodiment of the invention comprises clinical application of the information described herein to a patient. This can occur after a patient arrives in the clinic and presents with obesity symptoms or symptoms of a metabolic disorder. A non-limiting example of an effective dose range for a therapeutic compound described herein is from about 0.1 and 5,000 mg/kg of body weight/per day. One of ordinary skill in the art would be able to study the relevant factors and make the determination regarding the effective amount of the therapeutic compound without undue experimentation.

The therapeutic peptides described herein have been shown to be well tolerated and the symptoms were assessed using clinical scores criteria. The treatment protocol can also optionally include administration of effective amounts of one or more of therapeutic agents that treat or inhibit obesity. Such agents, include without limitation Bupropion-naltrexone. Liraglutide (Saxenda), Orlistat (Xenical, Alli), Wegovy, and Phentermine-topiramate. The treatment protocol can also optionally include lifestyle changes or surgeries that help with the management of weight gain.

While certain features of the invention have been described herein, many modifications, substitutions, changes, and equivalents will now occur to those of ordinary skill in the art. It is, therefore, to be understood that the appended claims are intended to cover all such modifications and changes as fall within the scope of the invention.

What is claimed is:

1. An isolated or purified peptide having the amino acid sequence of SEQ ID NO: 1 or SEQ ID NO: 2 or a functional sequence variant having at least 95% identity thereto.

2. The peptide according to claim 1, wherein said peptide has an anti-obesity activity.

3. The peptide of claim 1, comprising one or more modified amino acids selected from Gln(alkyn), Ala(Alkyn), Gly(Alkyn), Lys(N3) and the modified amino acids listed in Table 2 for bioconjugation including lipidation and/or fluorescent tagging.

4. The peptide of claim 3, wherein said modified amino acid facilitates bioconjugation of an agent selected from one or more of a N and, or C terminus protection moiety, a lipid, a recombinant FC-peptide, a cell penetrating peptide, and enhances one or more of peptide function, stability, and bioavailability.

5. A composition comprising the peptide of claim 1 and a pharmaceutically acceptable carrier.

6. The composition of claim 5 further comprising at least one of a HEPES buffer and acetate salt.

7. The composition of claim 5, wherein said composition is formulated to retain a neutral pH or said composition is formulated for intracerebroventricular injection or peripheral administration.

8. (canceled)

9. A nucleic acid sequence that encodes the peptide of claim 1.

10. The nucleic acid of claim 9, encapsulated in a liposome or an extracellular vesicle or affixed to a nanoparticle or lipid nanoparticle.

11. A vector comprising the nucleic acid of claim 9.

12. The vector of claim 11, wherein said vector is a plasmid vector, a lentiviral vector, or an AAV vector or said vector is. encapsulated in a liposome or an extracellular vesicle or affixed to a nanoparticle or lipid nanoparticle.

13. (canceled)

14. A method of treating obesity in a subject in need thereof, the method comprising administering an effective amount of the peptide of claim 1.

15. A method of treating a metabolic disease or disorder in a subject in need thereof, the method comprising administering an effective amount of the peptide of claim 1.

16. The method of claim 15, wherein the metabolic disease or disorder is selected from obesity, diabetes mellitus, type 1 diabetes, type 2 diabetes, dyslipidemia, insulin resistance, hepatic steatosis, hypercholesterolemia, and non-alcoholic fatty liver.

17. (canceled)

18. The method of claim 14, further comprising administering a second therapeutic agent that treats or inhibits obesity, or enforcing lifestyle interventions on diet and/or physical activity on said subject.

19. (canceled)

20. The method of claim 14, wherein the patient has a reduced food intake for 1, 3, 6, and/or 24 hours after administration of said peptide when compared to an untreated control or the weight of the patient decreases following administration of the peptide.

21. (canceled)

22. The method of claim 14, further comprising assessing the patient for a reduction in symptoms of obesity, diabetes mellitus, dyslipidemia, insulin resistance, hepatic steatosis, hypercholesterolemia, or non-alcoholic fatty liver.

23. The method of claim 14, wherein said administration is via route selected from systemic, intramuscular, topical, oral, parenteral, transdermal patch, aerosolized, pulmonary, ophthalmic, buccal, and lingually.

24. The method of claim 23, wherein said administration is via intracerebroventricular injection or peripheral administration.

25. The method of claim 18, wherein said peptide and said second therapeutic agent act synergistically to increase weight loss.
